(19) United States
(12) Patent Application Publication
(10) Pub. No.: US 2023/0107853 A1
(43) Pub. Date: Apr. 6, 2023

(54) METHOD FOR IDENTIFYING NEO-ANTIGENS

(71) Applicant: Oxford University Innovation Limited, Oxford (GB)

(72) Inventors: Ahmed Ashour AHMED, Oxford (Oxfordshire) (GB); Donatien CHEDOM-FOTSO, Oxford (Oxfordshire) (GB); Christopher YAU, Oxford (Oxfordshire) (GB); Gennaro PROTA, Oxford (Oxfordshire) (GB); Vincenzo CERUNDOLO, Oxford (GB); Uzi GILEADI, Oxford (Oxfordshire) (GB)

(21) Appl. No.: 17/910,841

(22) PCT Filed: Feb. 24, 2021
(86) PCT No.: PCT/GB2021/050456

§ 371 Date: Sep. 12, 2022

(30) Foreign Application Priority Data
Mar. 13, 2020 (GB) 2003669.5

(51) Int. Cl. A61K 39/00; A61K 35/17; C12N 5/0783; C12Q 1/6886

(52) U.S. Cl. CPC A61K 39/0011; A61K 35/17; C12N 5/0636; C12Q 1/6886

(57) ABSTRACT

The present invention relates to a process for identifying and/or isolating T cells which are capable of binding to a tumour in a mammalian subject having the tumour. Such T cells are of use in cancer immunotherapy. The invention also relates to a method of identifying a peptide neo-antigen for use in cancer immunotherapy in a mammalian subject having a tumour.

The present invention relates to a process for identifying and/or isolating T cells which are capable of binding to a tumour in a mammalian subject having the tumour. Such T cells are of use in cancer immunotherapy. The invention also relates to a method of identifying a peptide neo-antigen for use in cancer immunotherapy in a mammalian subject having a tumour.

T cell responses against human cancers contribute to the control of tumour growth, and targeting of CTLA-4 and the PD-1/PD-L1 axis has been very effective in enhancing anti-tumour immune responses, resulting in clinical objective responses [1, 2], particularly in patients with tumours expressing high mutational burden (high TMB) [1, 2]. These results underscore an unmet clinical need for many cancer patients with low TMB (i.e. the largest proportion of cancer patients), who could receive greater benefit from immune checkpoint inhibition treatment if optimal neo-epitopes can be identified and used in vaccination strategies.

To this end, new strategies need to be developed to identify the most immunogenic cancer-associated neo-epitopes. Current pipelines employed for neo-antigen prediction have resulted in a low rate of validation, suggesting that the current determinants of peptide immunogenicity are still suboptimal [3-5]. A greater understanding of the biology of the presentation of the cancer mutanome is thus needed in order to improve such algorithms.

Several parameters are currently considered when ranking the immunogenicity of mutations in cancer cells [6-8], but it remains unclear whether the subcellular localisation of tumour antigens can modulate the efficiency of priming of anti-tumour specific immune responses and whether this parameter should be considered in algorithms ranking immunogenicity of mutated peptides in cancer cells. This represents a critical knowledge gap, as current prognostic scores for responsiveness to immune checkpoint inhibitors are mainly based on the numbers of mutations, without taking into account whether the sub-cellular localisation of such mutated protein antigens may influence their ability to mount an immune response.

Tumour cells fail to directly prime tumour-specific immune responses, likely as a result of low co-stimulation; instead, dendritic cells (DCs) function simultaneously as both antigen-presenting cells (capable of taking up tumour debris) and as IL-12-producing cells, in a process referred to as ‘cross-priming’ [9, 10]. Consistent with a role for DCs in cross-priming tumour specific T cell responses, recent results have demonstrated the transfer of cellular components from tumour cells to antigen-presenting cells [9, 11], and in particular it has been shown that mitochondria can be transferred from tumours to DCs, via cytoplasts [11].

The inventors have recognised that this raises the possibility that mutations contained within mitochondrial-localised proteins in cancer cells could elicit specific strong T cell responses in vivo.

The mitochondrial DNA only encodes 13 proteins, which serve as subunits of the respiratory complexes [13]. However, mitochondria contain more than 1,200 proteins that are encoded by nuclear DNA and targeted to mitochondria via the expression of specific signalling motifs [14]. It is known that mutations in proteins encoded by mitochondrial genes can lead to the generation of CD8+ T cell responses [15, 16].

Previous studies have described the effect of subcellular localisation of protein antigens on direct presentation of T cell epitopes [17-20]. Furthermore, Yamazaki and colleagues have previously demonstrated that different portions of the antigenic protein LIVAT-BP are selected as antigenic epitopes for CD8+ T cell recognition depending on whether LIVAT-BP is located in the cytoplasm or in the mitochondria [20]. While these results are of interest, this paper falls short of distinguishing whether antigen location modulates: i) in vivo cross-priming of T cell responses; ii) proteasome-dependent degradation of the antigenic protein; and iii) tumour growth.

A process has now been developed for producing populations of T cells which are specific for cancer-associated tissue-specific neo-antigens, particularly for mitochondrial-specific neo-antigens. The process has been validated—clinically—in human samples by demonstrating the presence of CD8+ T cell responses specific to mitochondrial-localized neo-antigens in a cancer patient. Such T cells are particularly of use in cancer immunotherapy.

In one embodiment, the invention provides a process for identifying and/or isolating T cells which are capable of recognising cancer cells from a tumour in a mammalian subject having the tumour, the process comprising the steps:
(a) identifying a non-synonymous mutation in a polypeptide-encoding gene in the nuclear or mitochondrial genome of a cell in the subject's tumour tissue, wherein the mutation is one which is not present in the corresponding gene in the nuclear or mitochondrial genomes of cells of the subject's non-tumour tissues, and wherein the polypeptide is a mitochondrial polypeptide;
(b) identifying a plurality of fragments of the polypeptide which is encoded by the gene having the non-synonymous mutation, wherein each fragment has an amino acid sequence which spans the site of the mutated amino acid(s), and wherein each fragment is capable of being presented by a mammalian MHC1 molecule;
(c) contacting a population of cells from the subject with one or more of the plurality of fragments, wherein the population of cells comprises T cells; and
(d) identifying and/or isolating T cells from within the population of cells which recognise one or more of the plurality of fragments,
   wherein the T cells which are identified and/or isolated in Step (d) are T cells which are capable of recognising cancer cells from the tumour in the subject.

Preferably, the T cells are ones which are capable of specifically recognising cancer cells from the tumour. Preferably, the T cells are ones which are capable of recognising and killing cancer cells from the tumour. Preferably, the cancer cells are ones which express neo-antigens.

Step (a) may include obtaining samples of tumour tissue from the subject and/or samples of non-tumour tissues from the subject. Preferably, Step (a) is carried out by comparing nucleotide sequence data from samples of the subject's tumour and non-tumour tissues. Step (a) may additionally comprise the step of determining the expression level in the tumour tissue of mRNA transcripts from the polypeptide-encoding gene having the non-synonymous mutation.

Preferably, Step (d) is performed by (d) identifying and/or isolating T cells from within the population of cells which are capable of binding to a MHC1:fragment complex, wherein the fragment is one which is capable of stimulating the expansion of the T cells.

In another embodiment, the invention provides a method of identifying a peptide neo-antigen for use in cancer immunotherapy in a mammalian subject having the tumour, the method comprising the steps:
(a) identifying a non-synonymous mutation in a polypeptide-encoding gene in the nuclear or mitochondrial genome of a cell in the subject's tumour tissue, wherein the mutation is one which is not present in the corresponding gene in the nuclear or mitochondrial genomes of cells of the subject's non-tumour tissues, and wherein the polypeptide is a mitochondrial polypeptide;
(b) identifying a plurality of fragments of the polypeptide which is encoded by the gene having the non-synonymous mutation, wherein each fragment has an amino acid sequence which spans the site of the mutated amino acid(s), and wherein each fragment is capable of being presented by a mammalian MHC1 molecule;
(c) contacting a population of cells from the subject with one or more of the plurality of fragments, wherein the population of cells comprises T cells; and
(d) identifying fragments which are capable of stimulating T cells within the population of cells,

wherein fragments which are identified in Step (d) are peptides which are suitable for use in cancer immunotherapy in the mammalian subject having the tumour.

Preferably, the fragments are ones which are capable of specifically binding to the stimulated T cells when presented in a MHC1:fragment complex (e.g. in a tetramer assay).

The process of the invention is carried out in vitro or ex vivo.

The subject is a mammalian subject. The mammal may be human or non-human. For example, the subject may be a farm mammal (e.g. sheep, horse, pig, cow or goat), a companion mammal (e.g. cat, dog or rabbit) or a laboratory test mammal (e.g. mouse, rat or monkey). Preferably, the subject is a human. The human may, for example, be 0-10, 10-20, 20-30, 30-40, 40-50, 50-60, 60-70, 70-80, 80-90, 90-100 or above 100 years old. The human may be one who is suffering from or at risk from a particular disease or disorder.

The subject is one which has one or more tumours. The tumour may be a benign, pre-malignant or malignant tumour. The tumour may be a primary or secondary tumour. The tumour is preferably a solid tumour. The tumour comprises cancer cells. In some embodiments, the tumour is one whose size or carcinogenic (e.g. invasive) capabilities has previously been reduced. For example, the tumour may be one which has previously been at least partially resected (removed). Alternatively, or additionally, the tumour may be one which has previously been treated with another anti-tumour treatment, e.g. chemotherapy, immunotherapy or radiation, or a combination thereof.

Cancers are classified by the type of cell that the tumour cells resemble and is therefore presumed to be the origin of the tumour.

These types include:

Carcinoma: Cancers derived from epithelial cells. This group includes many of the most common cancers and include nearly all those in the breast, prostate, lung, pancreas and colon.

Sarcoma: Cancers arising from connective tissue (i.e. bone, cartilage, fat, nerve), each of which develops from cells originating in mesenchymal cells outside the bone marrow.

Germ cell tumour: Cancers derived from pluripotent cells, most often presenting in the testicle or the ovary (seminoma and dysgerminoma, respectively).

Blastoma: Cancers derived from immature “precursor” cells or embryonic tissue.

Brain and nervous system cancers include Astrocytoma, Brainstem glioma, Pilocytic astrocytoma, Ependymoma, Primitive neuroectodermal tumour, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma.

Breast cancers include Breast cancer, Invasive lobular carcinoma, Tubular carcinoma, Invasive cribriform carcinoma, Medullary carcinoma, Male breast cancer, Phyllodes tumour and Inflammatory Breast Cancer.

Endocrine system cancers include Adrenocortical carcinoma, Islet cell carcinoma (endocrine pancreas), Multiple endocrine neoplasia syndrome, Parathyroid cancer, Pheochromocytoma, Thyroid cancer and Merkel cell carcinoma.

Eye cancers include Uveal melanoma and Retinoblastoma.

Gastrointestinal cancers include Anal cancer, Appendix cancer, cholangiocarcinoma, Carcinoid tumour (gastrointestinal), Colon cancer, Colorectal cancer, Extrahepatic bile duct cancer, Gallbladder cancer, Gastric (stomach) cancer, Gastrointestinal carcinoid tumour, Gastrointestinal stromal tumour (GIST), Hepatocellular cancer, Pancreatic cancer (islet cell) and Rectal cancer.

Genitourinary and gynaecological cancers include Bladder cancer, Cervical cancer, Endometrial cancer, Extra-gonadal germ cell tumour, Ovarian cancer, Ovarian epithelial cancer (surface epithelial-stromal tumour), Ovarian germ cell tumour, Ovarian cancer Minimal Residual Disease (MRD), Penile cancer, Renal cell carcinoma, Renal pelvis and ureter, transitional cell cancer, Prostate cancer, Testicular cancer, Gestational trophoblastic tumour, Ureter and renal pelvis, transitional cell cancer, Urethral cancer, Uterine sarcoma, Vaginal cancer, Vulvar cancer and Wilms tumour.

Head and neck cancers include Esophageal cancer, Head and neck cancer, Head and neck squamous cell carcinoma (HNSCC), Nasopharyngeal carcinoma, Oral cancer, Oropharyngeal cancer, Paranasal sinus and nasal cavity cancer, Pharyngeal cancer, Salivary gland cancer and Hypopharyngeal cancer.

Skin cancers include Basal-cell carcinoma, Melanoma and Skin cancer (non-melanoma).

Thoracic and respiratory cancers include Bronchial adenomas/carcinoids, Small cell lung cancer, Mesothelioma, Non-small cell lung cancer, Pleuropulmonary blastoma, Laryngeal cancer, Thymoma and thymic carcinoma.

HIV/AIDS related cancers include AIDS-related cancers and Kaposi sarcoma.

In one particularly preferred embodiment, the tumour is an endometrioid, colon, melanoma, stomach or rectal tumour, most preferably an endometrioid tumour.

In another particularly preferred embodiment, the tumour is an ovarian tumour and the cancer cells are ovarian cancer minimal residual disease (MRD) cells.

Step (a) comprises:
(a) identifying a non-synonymous mutation in a polypeptide-encoding gene in the nuclear or mitochondrial genome of a cell in the subject's tumour tissue, wherein the mutation is one which is not present in the corresponding gene in the nuclear or mitochondrial genomes of cells of the subject's non-tumour tissues, and wherein the polypeptide is a mitochondrial polypeptide.

In this step, potential neo-antigens are identified. (Neo-antigens are antigens that are absent from the normal (wild-type) human genome.)

A non-synonymous mutation is a nucleotide mutation that alters the amino acid sequence of a polypeptide. In this case, the non-synonymous mutation is a mutation which alters the amino acid sequence of the mitochondrial polypeptide encoded by the gene. The mitochondrial polypeptide will therefore have a mutated amino acid sequence.

Depending on the extent of the mutation, one or more amino acids might be affected. The mutation may be an insertion, deletion or substitution. Preferably, the mutation is a single-nucleotide substitution.

Step (a) may include obtaining samples of tumour tissue from the subject and/or samples of non-tumour tissues from the subject. Preferably, the step does not include the step of obtaining samples of tumour tissue from the subject and/or samples of non-tumour tissues from the subject. In this latter case, identification is based on samples which have previously been obtained from the subject.

The mutation is one which is not present in the subject's non-tumour tissue. Preferably, the non-tumour tissue is a germ-line tissue. Preferably, the non-tumour tissue is blood or a normal (non-tumour) tissue adjacent to the tumour tissue.

It may be verified at this point that the sample of tumour tissue has a sufficient percentage of cancer cells to enable the subsequent analysis.

Preferably, Step (a) is carried out by comparing nucleotide sequence data from samples of the subject's tumour and non-tumour tissues. Nucleotide sequence data may be obtained from RNA or DNA extracted from the tumour and non-tumour tissues. Preferably, DNA from cells in the subject's tumour and non-tumour tissues is sequenced and compared in order to identify one or more non-synonymous mutations. DNA sequencing may be performed using any suitable method, e.g. using Illumina sequencing.

The polypeptide is a mitochondrial polypeptide, i.e. in cells, the polypeptide is located in mitochondria. As mentioned above, the mitochondrial genome only encodes 13 polypeptides, but mitochondria contain more than 1,200 polypeptides which are encoded by the nuclear genome and targeted to mitochondria via the expression of specific signalling motifs. As used herein, the term “mitochondrial polypeptide” encompasses both polypeptides which are encoded the mitochondrial genome and polypeptides which are encoded by the nuclear genome and which are subsequently targeted to mitochondria.

Step (a) may additionally comprise the step of determining the expression level in the tumour tissue of mRNA transcripts from the polypeptide-encoding gene having the non-synonymous mutation. In this way, it can be verified that the polypeptides which have been identified as ones having one or more mutations are actually expressed to a significant extent in the tumour tissue. RNA may be extracted from the tissues using any suitable method, e.g. using the Qiagen RNeasy Mini Kit. Quality may be assessed with the Agilent TapeStation.

Step (a) may also comprise the step of computing the percentage of cancer cells in a sample from the subject's tumour tissue that have the non-synonymous mutation.

Preferably, a mutation is selected which is present in a large fraction of the cancer cells in the tumour (e.g. at least 50%, 60%, 70%, 80% or 90%, preferably at least 70%), i.e. the mutation has high prevalence. This is known as prevalence estimation. Preferably, prevalence estimation is carried out using the OncoPhase technique (Chedom-Fotso et al. “OncoPhase: Quantification of somatic mutation cellular prevalence using phase information”, bioXRiv doi: https://doi.org/10.1101/046631).

Step (b) comprises:

(b) identifying a plurality of fragments of the polypeptide which is encoded by the gene having the non-synonymous mutation, wherein each fragment has an amino acid sequence which spans the site of the mutated amino acid(s), and wherein each fragment is capable of being presented by a mammalian MHC1 molecule.

After the non-synonymous mutations have been identified in the polypeptide-encoding gene, a plurality of test peptides are identified, each of which has a different amino acid sequence which spans the site of the mutation in the polypeptide. The peptides may be viewed as being fragments of the polypeptide, wherein each fragment comprises the mutated amino acid(s).

Preferably, the fragments are each 7-11 amino acids, more preferably 8-10 amino acids, in length. Preferably, the plurality of fragments is 1-45 fragments, more preferably 5-30 fragments.

The fragments which are produced are ones which are capable of being presented by MHC1 molecules. MHC class 1 molecules are major histocompatibility complex (MHC) molecules that are found on the cell surface of all nucleated cells in the bodies of mammals. Their function is to display peptide fragments of proteins from within the cell to cytotoxic T cells; this will trigger an immediate response from the immune system against a particular non-self antigen displayed with the help of an MHC class I protein.

The major histocompatibility complex (MHC) proteins in humans are encoded by the human leukocyte antigen (HLA) system or complex. In humans, the HLAs corresponding to MHC class 1 are HLA-A, HLA-B, and HLA-C. Methods for determining whether a test peptide is capable of binding to and be presented by MHC1 molecules are well known in the art (e.g. Stronen, E., et al., “Targeting of cancer neoantigens with donor-derived T cell receptor repertoires.” Science, 2016. 352(6291): pp. 1337-41). MHC class 1 molecules bind peptides that are predominantly 8-10 amino acid in length, although the binding of longer peptides has also been reported. The MHC class 1 molecule should be from the same species as the subject, e.g. both the MHC1 and subject are human.

Step (c) comprises:
(c) contacting a population of cells from the subject with one or more of the plurality of fragments, wherein the population of cells comprises T cells.

In this step, the one or more of the fragments which were identified in Step (b) are contacted with T cells from the subject.

The populations of cells from the subject comprising T cells are preferably obtained from a blood sample, e.g. a population of peripheral blood mononuclear cells (PBMCs). PBMCs can be extracted from whole blood using ficoll and gradient centrifugation, which will separate the blood into a top layer of plasma, followed by a layer of PBMCs and a bottom fraction of poly-morphonuclear cells (such as neutrophils and eosinophils) and erythrocytes.

Preferably, the T cells are CD8+ cells. In some embodiments, the population of cells comprising T cells consists substantially of T cells, preferably substantially of CD8+ T cells.

Each of the plurality of fragments may be contacted individually with a population of T cells (i.e. each different fragment is contacted with a population comprising T cells) or the fragments may be pooled together first (and then contacted with a population comprising T cells). For example, pools of 1-30, preferably 15-25 fragments, may be contacted with a population of cells comprising T cells.

Step (d) comprises:
d) identifying and/or isolating T cells from within the population of cells which recognise one or more of the plurality of fragments.

Within the population of cells from the subject comprising T cells, there may be:
(i) some T cells which will not recognise any of the plurality of fragments;
(ii) some T cells which recognise - for the first time - one or more of the plurality of fragments; and
(iii) some T cells which have previously been exposed to an antigen comprising one of the plurality of fragments.

Cells which fall into category (ii) and particularly category (iii) will be stimulated to divide and clonally expand, i.e. T cells which recognise one or more of the plurality of fragments will be stimulated by those fragments. Stimulated T cells may be identified by any suitable method, e.g. a MHC tetramer assay.

Preferably, Step (d) is performed by:
(d) identifying and/or isolating T cells from within the population of cells which are capable of binding to a MHC1:fragment complex, wherein the fragment is one which is capable of stimulating the expansion of the T cells.

For example, a tetramer assay (also known as a tetramer stain, REF 4) may be used to identify stimulated T cells. In such an assay, MHC tetramers which present individual fragments which were used to stimulate the T cells are contacted with cell populations comprising stimulated T cells; and those T cells to which the MHC:fragment tetramers bind are sorted and isolated, e.g. by FACS assay.

T cells which are identified and/or isolated in Step (d) are T cells which are capable of binding to the cancer cells in the tumour in the subject. T cells which are identified and/or isolated in Step (d) are T cells which are capable of recognising cancer cells from the tumour in the subject. Preferably, the T cells are ones which are capable of specifically recognising cancer cells from the tumour. Preferably, the T cells are ones which are capable of recognising and killing cancer cells from the tumour. Preferably, the cancer cells are ones which express neo-antigens

The ability of the stimulated T cells to kill cells MHC:fragment-presenting cells may be assayed using autologous Epstein-Barr virus-transformed lymphoblastoid cell lines (EBV-LCLs) generated from the subject's PBMCs, e.g. as described in the Examples herein.

In yet another embodiment, the invention provides T cells obtained or obtainable by a process of the invention.

The fragment or fragments which are capable of stimulating the expansion of the T cells may be identified. Such fragments may be deemed to be ones which mimic neo-antigens in the tumour. Such fragments may be used in vaccines against the tumour or in cancer immunotherapy.

In yet another embodiment, therefore, the invention provides a method of identifying a peptide neo-antigen for use in cancer immunotherapy in a mammalian subject having a tumour, the method comprising the steps:
(a) identifying a non-synonymous mutation in a polypeptide-encoding gene in the nuclear or mitochondrial genome of a cell in the subject's tumour tissue, wherein the mutation is one which is not present in the corresponding gene in the nuclear or mitochondrial genomes of cells of the subject's non-tumour tissues, and wherein the polypeptide is a mitochondrial polypeptide;
(b) identifying a plurality of fragments of the polypeptide which is encoded by the gene having the non-synonymous mutation, wherein each fragment has an amino acid sequence which spans the site of the mutated amino acid(s), and wherein each fragment is capable of being presented by a mammalian MHC1 molecule;
(c) contacting a population of cells from the subject with one or more of the plurality of fragments, wherein the population of cells comprises T cells; and
(d) identifying fragments which are capable of stimulating T cells within the population of cells,

wherein fragments which are identified in Step (d) are peptides which are suitable for use in cancer immunotherapy in the mammalian subject having the tumour.

Preferably, the fragments are ones which are capable of specifically binding to the stimulated T cells when presented in a MHC1:fragment complex (e.g. in a tetramer assay).

In a further embodiment, the invention provides a vaccine composition comprising a (peptide) fragment which has been identified by a method of the invention, optionally together with one or more pharmaceutically-acceptable adjuvants, carriers or diluents.

Methods for the culturing of cells and other recombinant molecular biology techniques are well known in the art (e.g. “Molecular Cloning: A Laboratory Manual” (Fourth Edition), Green, M R and Sambrook, J., (updated 2014)).

The disclosure of each reference set forth herein is specifically incorporated herein by reference in its entirety.

### BRIEF DESCRIPTION OF THE FIGURES

FIG. 1. Identification of CD8+ T cell clones specific for mitochondrial proteins in a cancer patient

(A) Scatter plot shows the disperse frequencies of nonsynonymous somatic mutations in mitochondrial proteins that are expressed by gDNA from 9,508 samples across 31 cancer types from The Cancer Genome Atlas (TCGA, version 02-04-2018). The total number of nonsynonymous somatic mutations in mitochondria-localised proteins is 93,679. The x axis represents the number of such mutations per patient. The top five cancer types with the highest average frequency are endometrioid, colon, melanoma, stomach and rectal in the decreasing order. The somatic mutations are the output of MuTect2 [49] with hg38 assembly.

(B) By comparing the sequences obtained from tumour and normal DNA, tumour-specific non-synonymous single-nucleotide variations (SNV) were identified. A computational pipeline was used to examine the mutant peptide regions for binding to the patient's HLA alleles. We then divided the epitopes based on whether they derived from proteins localised in mitochondria or cytosol by employing existing databases, and we synthesized 60 peptides for each group of proteins. Peptide—HLA-A2 complexes were generated employing the UV-mediated ligand exchange technology [50]. After restimulation, clones specific for mitochondrial localised (left panel, (i)) and non-mitochondrial-localised proteins (right panel, (ii)) were identified. The ability of identified CD8+ T cell clones to kill peptide pulsed autologous EBV immortalised B cell lines (C) and to produce IFNγ was investigated (D).

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Non-synonymous Somatic Mutations in Endometrial Cancer Patients

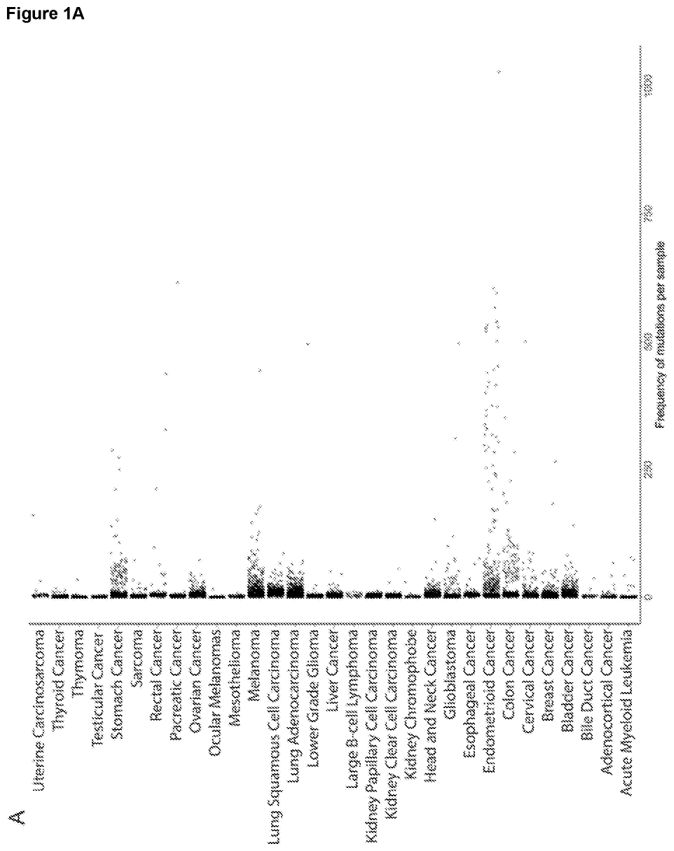

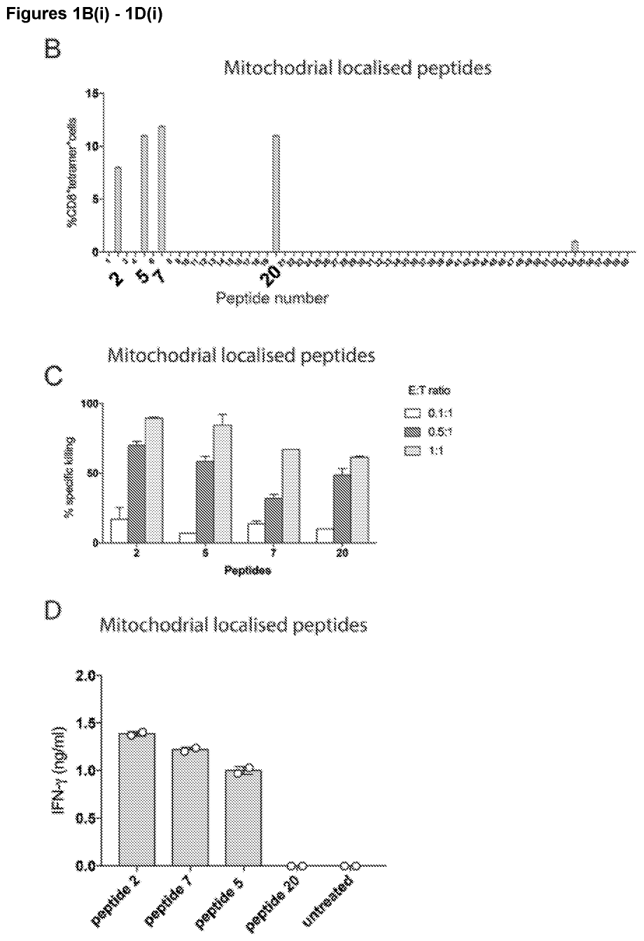

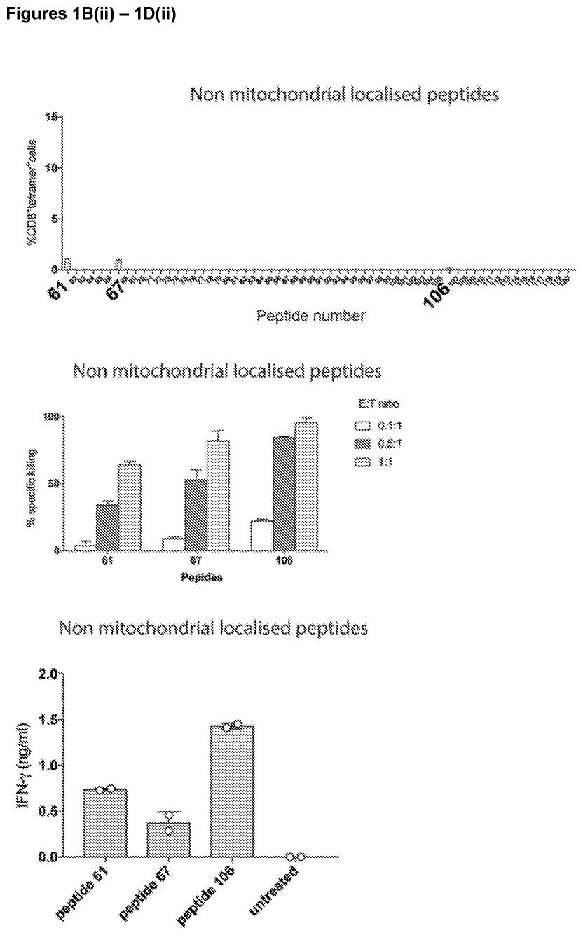

The presence of CD8+ T cells specific for mutated mitochondrial localised proteins was investigated in human cancers. The frequencies of non-synonymous somatic mutations in mitochondrial proteins that are expressed by genomic DNA from 9,508 samples across 31 cancer types from The Cancer Genome Atlas (TCGA, version 02-04-2018) were investigated. We observed the presence of mutations of mitochondrial-localised proteins across different tumour types, with the highest average frequency present in endometrioid cancers (FIG. 1A). We therefore focused our studies on endometrial cancer patients, and studied the immune response in one patient, with a hyper-mutated phenotype caused by the loss of function of the proof reading DNA polymerase epsilon (POLE) [38].

### Example 2: Neo-antigen Prediction and Selection of Peptides for Screening

By comparing the sequences obtained from tumour and germline DNA, tumour-specific non-synonymous single-nucleotide variations (SNV) were identified

Endometrial tumour tissue and blood collection were obtained from a patient recruited to the Gynaecological Oncology Targeted Therapy Study 01 (GO-Target-01) under research ethics approval number 11-SC-0014. The patient gave informed consent. Whole-genome sequencing was performed on blood and tumour tissues (BGI Tech Solutions Ltd, Hong Kong, PR China) as previously described [S1].

RNA sequencing was performed to check the expression of potential neo-epitopes. RNA was extracted using the Qiagen RNeasy Mini Kit and its quality assessed with the Agilent TapeStation before preparing the sequencing libraries. Two technical replicates were prepared from 400 ng RNA each using the NEBNext Ultra Directional RNA Library Prep Kit for Illumina (E7420) in combination with the NEBNext Poly(A) mRNA Magnetic Isolation Module (E7490) and NEBNext Q5 Hot Start HiFi PCR Master Mix (M0543).

The libraries were indexed and enriched by 14 cycles of amplification, assessed using the Agilent TapeStation and then quantified by Qubit. Multiplexed library pools were quantified with the KAPA Library Quantification Kit (KK4835) and sequenced using 80bp PE reads on the Illumina NextSeq500 platform.

A computational pipeline was used to examine predicted mutant peptide regions for binding to the patient's HLA allele HLAA*02:01. Tumour-specific non-synonymous mutations were predicted and ranked as previously shown [S2].

We then divided prioritised epitopes based on their mitochondrial or non-mitochondrial localisation and synthesized 60 peptides for each location group. Peptides were synthesized by Pepscan Presto BV (The Netherlands).

### Example 3: Expansion of Antigen-specific T Cells

The patient's PBMC were stimulated and expanded in the presence of each peptide.

120 peptides were pooled in 6 groups of 20 peptides each. In vitro stimulation of CD8 T cells was done as previously described [S3]. Briefly, 5-8×10⁶ peripheral blood mononuclear cells (PBMCs) from the patient were stimulated with each peptide pool at a final concentration of 20 μg/mL (2 μg/mL, each peptide) for 3 days in RH10 (RPMI with 10% heat-inactivated human serum (Sigma), 2 mM L-glutamine, 10 mM HEPES, 1 mM sodium pyruvate, non-essential amino acids (1×), penicillin (25,000 U), streptomycin (25 mg), 50 μM (β-mercaptoethanol (Gibco)) supplemented with 25 ng/mL human IL-7 (Peprotech). On day 3, half of the media was replaced with RH10-IL2 (RH10 supplemented with 1000 IU human IL-2 (Novartis)). When confluent, cells were split using RH10-IL2. To further increase cell expansion, 4 weeks later, cells were re-stimulated with phyto-haemagglutinin 1 μg/mL in RH10-IL2 and in the presence of irradiated PBMC feeders and rest for 3-4 weeks before screening.

### Example 4: Neo-antigen-specific T Cells Screening

HLA-A2 tetramers loaded with mitochondrial and cytosolic derived peptides were used to assess the presence of neo-antigen-specific T cells in the expanded PBMCs.

Generation of peptide-MHC class I monomers and tetramerization was performed as previously described [S4]. Briefly, biotin-tagged HLA-A2 complexes were folded with the UV-sensitive peptide KILGFVFLV (SEQ ID NO: 1). 2 μg of HLA-A2 complexes were UV-exchanged for 1 hour with each screening peptide at a final concentration of 200 μg/mL, in 20 μL. After centrifugation at 2250 g, 1.5 μg of complexes (15 μL supernatant) were collected and tetramerized with 1.5 μL of a 1:1 mix streptavidin-APC/streptavidin-PE (eBioscience). Free biotin in the complexes was blocked by adding 20 μL of 50 μM D-biotin. 1×10⁵ stimulated PBMCs were incubated in 50 ul staining buffer (PBS 0.5% BSA) containing 2.5 μL of multimers, for 30 minutes at 37° C. Cells were washed two times with staining buffer and stained with LIVE/DEAD Fixable Aqua (Thermo Fisher), anti-CD3 FITC (clone SK7, Biolegend), and anti-CD8 PerCP-Cy5.5 (clone SK1, Biolegend). Cells were analysed in a BD LSR Fortessa instrument. PE and APC tetramer positive cells were sorted in a BD Fusion instrument and further expanded for functional assays.

### Example 5: VITAL Assay

A VITAL assay was used to determine the cell-killing capacity of the identified CD8+ T cells.

Autologous Epstein-Barr virus-transformed lymphoblastoid cell lines (EBV-LCLs) were generated from PBMCs, using supernatant of EBV producing B95-8 marmoset cells and 2 μg/mL cyclosporin A (Sigma). EBV-LCLs used as target cells were loaded with 1 μM peptides, for 1 hour at 37° C. Loaded and non-loaded cells were stained with either CellTrace Far Red or CellTracker Orange CMTMR dyes (Thermo Fisher) and quenched with FCS. After two wash cycles, loaded and non-loaded targets were mixed in a 1:1 ratio and plated in a 96-U-bottom well plate. Effector T cells, previously incubated overnight in the absence of IL-2, were added to the wells at the indicated effector-to-target ratio, in duplicates. Following 4.30 hours incubation at 37° C., cells were stained with LIVE/DEAD Fixable Aqua (Thermo Fisher) and anti-CD8-FITC (clone SK1, Biolegend). Cells were analysed in a BD LSR Fortessa instrument.

For the ELISAs, EBV-LCLs were loaded with 1 μM peptide, for 1 hour at 37° C., and washed twice. Loaded cells were plated at 25,000 cells per well in a U bottom 96-well plate and used to stimulate 2,500 T cells, in duplicates. Following 16 hours incubation, the production of IFNγ was assessed by ELISA (BD Pharmingen).

We identified CD8+ T cells recognising 4 neo-antigens derived from 2 mitochondrial-localised mutated proteins and 3 neo-antigens derived from proteins localised in the cytosol (FIG. 1B and Table 1, below), which were capable of specifically killing peptide-pulsed autologous EBV-LCLs (FIG. 1C) and producing IFNγ (FIG. 1D).

TABLE 1
Peptide sequences of the identified immunogenic mutated
peptides (SEQ ID NOs: 2-8)

| Peptide ID | Necepitope | Gene | Cellular localization |
|---|---|---|---|
| 2 | LLQFYIPEV | NFU1 | Mitochondria |
| 5 | NLLQFYIPEV | NMU1 | |
| 7 | GLMGAGITQV | HADHA | |
| 20 | LLQFYIPEVE | NPU1 | |
| 61 | FLPELIFGL | MALL | Non Mitochondria |
| 67 | FFLPELIFGL | MALL | |
| 106 | KLIHDLLTI | EIF3M | |

### REFERENCES

1. Yarchoan, M., A. Hopkins, and E. M. Jaffee, Tumor Mutational Burden and Response Rate to PD-1 Inhibition. N Engl J Med, 2017. 377(25): p. 2500-2501.

2. Schumacher, T. N. and R. D. Schreiber, Neoantigens in cancer immunotherapy. Science, 2015. 348(6230): p. 69-74.

3. The problem with neoantigen prediction. Nat Biotechnol, 2017. 35(2): p. 97.

4. Vitiello, A. and M. Zanetti, Neoantigen prediction and the need for validation. Nat Biotechnol, 2017. 35(9): p. 815-817.

5. Lee, C. H., et al., Update on Tumor Neoantigens and Their Utility: Why It Is Good to Be Different. Trends Immunol, 2018. 39(7): p. 536-548.

6. Bjerregaard, A. M., et al., MuPeXI: prediction of neo-epitopes from tumor sequencing data. Cancer Immunol Immunother, 2017. 66(9): p. 1123-1130.

7. Zhou, Z., et al., TSNAD: an integrated software for cancer somatic mutation and tumour-specific neoantigen detection. R Soc Open Sci, 2017. 4(4): p. 170050.

8. Kim, S., et al., Neopepsee: accurate genome-level prediction of neoantigens by harnessing sequence and amino acid immunogenicity information. Ann Oncol, 2018. 29(4): p. 1030-1036.

9. Roberts, E. W., et al., Critical Role for CD103(+)/CD141(+) Dendritic Cells Bearing CCR7 for Tumor Antigen Trafficking and Priming of T Cell Immunity in Melanoma. Cancer Cell, 2016. 30(2): p. 324-336.

10. Cruz, F.M., et al., The Biology and Underlying Mechanisms of Cross-Presentation of Exogenous Antigens on MHC-I Molecules. Annu Rev Immunol, 2017. 35: p. 149-176.

11. Headley, M. B., et al., Visualization of immediate immune responses to pioneer metastatic cells in the lung. Nature, 2016. 531(7595): p. 513-7.

12. Xu, M. M., et al., Dendritic Cells but Not Macrophages Sense Tumor Mitochondrial DNA for Cross-priming through Signal Regulatory Protein alpha Signaling. Immunity, 2017. 47(2): p. 363-373 e5.

13. Enriquez, J.A., Supramolecular Organization of Respiratory Complexes. Annu Rev Physiol, 2016. 78: p. 533-61.

14. Wiedemann, N. and N. Pfanner, Mitochondrial Machineries for Protein Import and Assembly. Annu Rev Biochem, 2017. 86: p. 685-714.

15. Fischer Lindahl, K., et al., Molecular definition of a mitochondrially encoded mouse minor histocompatibility antigen. Cold Spring Harb Symp Quant Biol, 1989. 54 Pt 1: p. 563-9.

16. Pierini, S., et al., A Tumor Mitochondria Vaccine Protects against Experimental Renal Cell Carcinoma. J Immunol, 2015. 195(8): p. 4020-7.

17. Shen, L. and K. L. Rock, Cellular protein is the source of cross-priming antigen in vivo. Proc Natl Acad Sci U S A, 2004. 101(9): p. 3035-40.

18. Rimoldi, D., et al., Subcellular localization of the melanoma-associated protein Melan-AMART-1 influences the processing of its HLA-A2-restricted epitope. J Biol Chem, 2001. 276(46): p. 43189-96.

19. Matheoud, D., et al., Parkinson's Disease-Related Proteins PINK1 and Parkin Repress Mitochondrial Antigen Presentation. Cell, 2016. 166(2): p. 314-327.

20. Yamazaki, H., et al., Epitope selection in major histocompatibility complex class I mediated pathway is affected by the intracellular localization of an antigen. Eur J Immunol, 1997. 27(2): p. 347-53.

21. Jin, L., et al., MPYS is required for IFN response factor 3 activation and type I IFN production in the response of cultured phagocytes to bacterial second messengers cyclic-di-AMP and cyclic-di-GMP. J Immunol, 2011. 187(5): p. 2595-601.

22. Bridgeman, A., et al., Viruses transfer the antiviral second messenger cGAMP between cells. Science, 2015. 349(6253): p. 1228-32.

23. Michallet, M. C., et al., TRADD protein is an essential component of the RIG-like helicase antiviral pathway. Immunity, 2008. 28(5): p. 651-61.

24. Porgador, A., et al., Localization, quantitation, and in situ detection of specific peptide-MHC class I complexes using a monoclonal antibody. Immunity, 1997. 6(6):p. 715-26.

25. Fernandez-Vizarra, E., et al., Isolation of mitochondria for biogenetical studies: An update. Mitochondrion, 2010. 10(3): p. 253-62.

26. Choi, E. M., et al., High avidity antigen-specific CTL identified by CD8-independent tetramer staining. J Immunol, 2003. 171(10): p. 5116-23.

27. Palmowski, M. J., et al., Intravenous injection of a lentiviral vector encoding NY-ESO-1 induces an effective CTL response. J Immunol, 2004. 172(3): p. 1582-7.

28. Norbury, C. C., et al., CD8+ T cell cross-priming via transfer of proteasome substrates. Science, 2004. 304(5675): p. 1318-21.

29. Basta, S., et al., Cross-presentation of the long-lived lymphocytic choriomeningitis virus nucleoprotein does not require neosynthesis and is enhanced via heat shock proteins. J Immunol, 2005. 175(2): p. 796-805.

30. Townsend, A., et al., Defective presentation to class I-restricted cytotoxic T lymphocytes in vaccinia-infected cells is overcome by enhanced degradation of antigen. J Exp Med, 1988. 168(4): p. 1211-24.

31. Yewdell, J. W., U. Schubert, and J. R. Bennink, At the crossroads of cell biology and immunology: DRiPs and other sources of peptide ligands for MHC class I molecules. J Cell Sci, 2001. 114(Pt 5): p. 845-51.

32. Woo, S. R., et al., STING-dependent cytosolic DNA sensing mediates innate immune recognition of immunogenic tumors. Immunity, 2014. 41(5): p. 830-42.

33. Deng, L., et al., STING-Dependent Cytosolic DNA Sensing Promotes Radiation-Induced Type I Interferon-Dependent Antitumor Immunity in Immunogenic Tumors. Immunity, 2014. 41(5): p. 843-52.

34. Hildner, K., et al., Batf3 deficiency reveals a critical role for CD8alpha+ dendritic cells in cytotoxic T cell immunity. Science, 2008. 322(5904): p. 1097-100.

35. Sancho, D., et al., Identification of a dendritic cell receptor that couples sensing of necrosis to immunity. Nature, 2009. 458(7240): p. 899-903.

36. Guo, C., et al., Absence of scavenger receptor A promotes dendritic cell-mediated cross-presentation of cell-associated antigen and antitumor immune response. Immunol Cell Biol, 2012. 90(1): p. 101-8.

37. Lower, M., et al., Confidence-based somatic mutation evaluation and prioritization. PLoS Comput Biol, 2012. 8(9): p. e1002714.

38. Temko, D., et al., Somatic POLE exonuclease domain mutations are early events in sporadic endometrial and colorectal carcinogenesis, determining driver mutational landscape, clonal neoantigen burden and immune response. J Pathol, 2018. 245(3): p. 283-296.

39. Balachandran, V.P., et al., Identification of unique neoantigen qualities in long-term survivors of pancreatic cancer. Nature, 2017. 551(7681): p. 512-516.

40. Luksza, M., et al., A neoantigen fitness model predicts tumour response to checkpoint blockade immunotherapy. Nature, 2017. 551(7681): p. 517-520.

41. Chatterjee, B., et al., Internalization and endosomal degradation of receptor-bound antigens regulate the efficiency of cross presentation by human dendritic cells. Blood, 2012. 120(10): p. 2011-20.

42. Gros, M. and S. Amigorena, Regulation of Antigen Export to the Cytosol During Cross-Presentation. Front Immunol, 2019. 10: p. 41.

43. Delamarre, L., et al., Differential lysosomal proteolysis in antigen-presenting cells determines antigen fate. Science, 2005. 307(5715): p. 1630-4.

44. Lennon-Dumenil, A. M., et al., Analysis of protease activity in live antigen-presenting cells shows regulation of the phagosomal proteolytic contents during dendritic cell activation. J Exp Med, 2002. 196(4): p. 529-40.

45. Savina, A., et al., NOX2 controls phagosomal pH to regulate antigen processing during crosspresentation by dendritic cells. Cell, 2006. 126(1): p. 205-18.

46. Mantegazza, A. R., et al., NADPH oxidase controls phagosomal pH and antigen cross-presentation in human dendritic cells. Blood, 2008. 112(12): p. 4712-22.

47. Accapezzato, D., et al., Chloroquine enhances human CD8+ T cell responses against soluble antigens in vivo. J Exp Med, 2005. 202(6): p. 817-28.

48. Shi, Y., J. E. Evans, and K. L. Rock, Molecular identification of a danger signal that alerts the immune system to dying cells. Nature, 2003. 425(6957): p. 516-21.

49. Cibulskis, K., et al., Sensitive detection of somatic point mutations in impure and heterogeneous cancer samples. Nat Biotechnol, 2013. 31(3): p. 213-9.

50. Rodenko, B., et al., Generation of peptide-MHC class I complexes through UVmediated ligand exchange. Nat Protoc, 2006. 1(3): p. 1120-32.

### Supplemental References

S1. Temko, D., et al., Somatic POLE exonuclease domain mutations are early events in sporadic endometrial and colorectal carcinogenesis, determining driver mutational landscape, clonal neoantigen burden and immune response. J Pathol, 2018. 245(3): p. 283-296.

S2. Stronen, E., et al., Targeting of cancer neoantigens with donor-derived T cell receptor repertoires. Science, 2016. 352(6291): p. 1337-41.

S3. Chen, J. L., et al., NY-ESO-1 specific antibody and cellular responses in melanoma patients primed with NY-ESO-1 protein in ISCOMATRIX and boosted with recombinant NY-ESO-1 fowlpox virus. Int J Cancer, 2015. 136(6): p. E590-601.

S4. Rodenko, B., et al., Generation of peptide-MHC class I complexes through UV mediated ligand exchange. Nat Protoc, 2006. 1(3): p. 1120-32.

S5. Palmowski, M. J., et al., Role of immunoproteasomes in cross-presentation. J Immunol, 2006. 177(2): p. 983-90.

S6. Denkberg, G., et al., Phage display-derived recombinant antibodies with TCR-like specificity against alpha-galactosylceramide and its analogues in complex with human CD1d molecules. Eur J Immunol, 2008. 38(3): p. 829-40.

### Sequence Listing Free Text

<210>1 <223>UV-sensitive peptide

<210>2 <223>Neo-antigen

<210>3 <223>Neo-antigen

<210>4 <223>Neo-antigen

<210>5 <223>Neo-antigen

<210>6 <223>Neo-antigen

<210>7 <223>Neo-antigen

<210>8 <223>Neo-antigen

1. A process for identifying and/or isolating T cells which are capable of recognising cancer cells from a tumour in a mammalian subject having the tumour, the process comprising the steps:
(a) identifying a non-synonymous mutation in a polypeptide-encoding gene in the nuclear or mitochondrial genome of a cell in the subject's tumour tissue, wherein the mutation is one which is not present in the corresponding gene in the nuclear or mitochondrial genomes of cells of the subject's non-tumour tissues, and wherein the polypeptide is a mitochondrial polypeptide;
(b) identifying a plurality of fragments of the polypeptide which is encoded by the gene having the non-synonymous mutation, wherein each fragment has an amino acid sequence which spans the site of the mutated amino acid(s), and wherein each fragment is capable of being presented by a mammalian MHC1 molecule;
(c) contacting a population of cells from the subject with one or more of the plurality of fragments, wherein the population of cells comprises T cells; and
(d) identifying and/or isolating T cells from within the population of cells which recognise one or more of the plurality of fragments, wherein the T cells which are identified and/or isolated in Step (d) are T cells which are capable of recognising cancer cells from the tumour in the subject.

2. The A process as claimed in claim 1, wherein the mammalian subject is human.

3. The A process as claimed in claim 1, wherein:
(i) the tumour is an endometrioid, colon, melanoma, stomach or rectal tumour; or
(ii) wherein the tumour is an ovarian tumour and the cancer cells are ovarian cancer minimum residual disease (MRD) cells.

4. The A process as claimed in claim 1, wherein the mutation is a single-nucleotide substitution.

5. The A process as claimed in claim 1, wherein the non-tumour tissue is blood or a normal tissue adjacent to the tumour tissue.

6. The A process as claimed in claim 1, wherein Step (a) is carried out by comparing nucleotide sequence data from samples of the subject's tumour and non-tumour tissues in order to identify one or more non-synonymous mutations.

7. The process as claimed in claim 1, wherein Step (a) additionally comprises the step:
determining the expression level in the tumour tissue of mRNA transcripts from the polypeptide-encoding gene having the non-synonymous mutation.

8. The process as claimed in claim 1, wherein Step (a) additionally comprises the step:
computing the percentage of cancer cells in a sample from the subject's tumour tissue that have the non-synonymous mutation.

9. The process as claimed in claim 1, wherein in Step (b) the fragments are each 7-11 amino acids in length.

10. The A process as claimed in claim 1, wherein in Step (c) the T cells are CD8+ cells.

11. The process as claimed in claim 1, wherein Step (d) comprises:
(d) identifying and/or isolating T cells from within the population of cells which are capable of binding to a MHC1:fragment complex, wherein the fragment is one which is capable of stimulating the expansion of the T cells.

12. The process as claimed in claim 1, wherein in Step (d),
the stimulated T cells are identified by a MHC tetramer assay.

13. The process as claimed in claim 1, wherein in Step (d), the stimulated T cells are isolated by FACS.

14. T cells obtained or obtainable by a process as claimed in claim 1.

15. A method of identifying a peptide neo-antigen for use in cancer immunotherapy in a mammalian subject having the tumour, the method comprising the steps:
(a) identifying a non-synonymous mutation in a polypeptide-encoding gene in the nuclear or mitochondrial genome of a cell in the subject's tumour tissue, wherein the mutation is one which is not present in the corresponding gene in the nuclear or mitochondrial genomes of cells of the subject's non-tumour tissues, and wherein the polypeptide is a mitochondrial polypeptide;
(b) identifying a plurality of fragments of the polypeptide which is encoded by the gene having the non-synonymous mutation, wherein each fragment has an amino acid sequence which spans the site of the mutated amino acid(s), and wherein each fragment is capable of being presented by a mammalian MHC1 molecule;
(c) contacting a population of cells from the subject with one or more of the plurality of fragments, wherein the population of cells comprises T cells; and
(d) identifying fragments which are capable of stimulating T cells within the population of cells,
wherein fragments which are identified in Step (d) are peptides which are suitable for use in cancer immunotherapy in the mammalian subject having the tumour.

16. A vaccine composition comprising a (peptide) fragment which has been identified by a method as claimed in claim 15, optionally together with one or more pharmaceutically-acceptable adjuvants, carriers or diluents.


SEQUENCE LISTING (8 sequences)

SEQ ID NO: 1 - protein, 9 aa, Artificial Sequence
  UV-sensitive peptide
KILGFVFLV

SEQ ID NO: 2 - protein, 9 aa, Artificial Sequence
  Neo-antigen
LLQFYIPEV

SEQ ID NO: 3 - protein, 10 aa, Artificial Sequence
  Neo-antigen
NLLQFYIPEV

SEQ ID NO: 4 - protein, 10 aa, Artificial Sequence
  Neo-antigen
GLMGAGITQV

SEQ ID NO: 5 - protein, 10 aa, Artificial Sequence
  Neo-antigen
LLQFYIPEVE

SEQ ID NO: 6 - protein, 9 aa, Artificial Sequence
  Neo-antigen
FLPELIFGL

SEQ ID NO: 7 - protein, 10 aa, Artificial Sequence
  Neo-antigen
FFLPELIFGL

SEQ ID NO: 8 - protein, 9 aa, Artificial Sequence
  Neo-antigen
KLIHDLLTI